(19) United States
(12) Patent Application Publication
(10) Pub. No.: US 2007/0231407 A1
(43) Pub. Date: Oct. 4, 2007

(54) Method of treating gallium-nitrate resistant tumors using gallium-containing compounds

(76) Inventor: Christopher R. Chitambar, Milwaukee, WI (US)

Correspondence Address: QUARLES & BRADY LLP, 33 E. MAIN ST, SUITE 900, P.O BOX 2113, MADISON, WI, 53701-2113, US

(21) Appl. No.: 11/732,228

(22) Filed: Apr. 3, 2007

Related U.S. Application Data
(60) Provisional application No. 60/788,957, filed on Apr. 4, 2006.

(51) Int. Cl. A61K 38/16; A61K 33/24; A61K 31/555

(52) U.S. Cl. 424/617; 514/6; 514/184

(57) ABSTRACT

The present invention relates to novel methods of using gallium containing compounds to treat mammals with gallium nitrate-resistant tumors.

### CROSS-REFERENCE TO RELATED APPLICATIONS

This application claims the benefit of U.S. Provisional Application No. 60/788,957 filed Apr. 4, 2006. This application is incorporated herein by reference in its entirety.

### STATEMENT REGARDING FEDERALLY SPONSORED RESEARCH OR DEVELOPMENT

This invention was made with United States government support awarded by the following agency: USPHS Grant No. RO1CA109518. The United States has certain rights in this invention.

### BACKGROUND OF THE INVENTION

Non-radioactive gallium compositions and compositions containing other Group IIIa elements have been found effective in treating some mammalian tumors. Gallium is thought to be the least toxic and most effective of these Group IIIa elements (Hart and Adamson, (1971) PNAS; 68(7): 1623-1626). It is known that Gallium becomes concentrated in malignant tumors and sites of infection. Gallium also appears to favorably impact calcium deposition, making bones more resistant to degradation caused by cancer metastasis. In 1991, intravenously administered gallium nitrate was approved in the U.S. for the treatment of hypercalcemia of malignancy.

In recent years, various forms of gallium-containing compounds have been used as antineoplastic agents as described in Chitambar, C. (2004) Current Opinion in Oncology 16(6):547-552, incorporated by reference here in its entirety. In published clinical studies, intravenously administered group IIIa metal salt gallium nitrate (GaN) demonstrated preliminary evidence of anti-tumor activity in several cancer indications, including multiple myeloma, lymphoma and bladder cancer (see Stauss, D J. (2003) Semin Oncol 2 Suppl. 5: 25-33). Recent clinical trials have demonstrated that FDA approved GaN has significant activity in non-Hodgkin's lymphoma (NHL). GaN also has efficacy in the treatment of NHL and is being used to treat such patients. However, the mode of administration for GaN is inefficient and cumbersome. For example, GaN is administered to patients slowly by continuous intravenous infusion, over a 5 to 7 day period.

In addition to GaN, it has been reported that other gallium-containing compounds, such as gallium (III) complexes of 3-hydroxy-4-pyrones may provide physiologically active gallium levels in mammals for a variety of medical and veterinary applications (U.S. Pat. No. 6,004,951, incorporated by reference here in its entirety). These gallium compounds may also be capable of treating specific types of gallium-responsive cancers, such as refractory lymphoma (U.S. Pat. No. 6,087,354, incorporated by reference here in its entirety). Most notably, gallium maltolate (GaM), a neutral 3:1 (3-hydroxy-2-methyl-4-pyrone:gallium) complex has shown promise as a chemotherapeutic agent for treatment of hepatocellular carcinoma (see Chua M S, et al. (2006) Anticancer Research 26:1739-1744).

Despite advances in therapy for lymphoma, particularly, non-Hodgkin's lymphoma (NHL), the mortality from this disease remains high because gallium-containing compounds do not work for all lymphoma patients. In fact, a significant fraction of lymphoma patients treated with GaN fail to respond due to tumor cell resistance to this drug. With all of the research in this field, still little is understood about the mechanism of tumor resistance to GaN (Bernstein L R. Pharmacol Rev 1998; 50:665-82, incorporated by reference here in its entirety). Hence, there is a great need to understand why a segment of the population that has lymphoma does not respond to GaN treatment and to develop new pharmaceutical drugs to treat this malignancy. Therefore, alternative compositions effective for treatment of lymphoma, particularly those forms that fail to respond to GaN would be a desirable contribution to the art.

### BRIEF SUMMARY OF THE INVENTION

The present invention is broadly summarized as a novel method for inhibiting the growth of a diverse array of gallium nitrate-resistant tumor cells in mammals by contacting such cells with an effective amount of a gallium-containing compound. The invention is based upon the discovery that certain tumor cells are not responsive to GaN, but are responsive to other gallium (III) complexes. Accordingly, the methods of the invention broadly relate to using a gallium (III) complex or derivatives thereof described herein to effectively inhibit growth of tumor cells not responsive to GaN.

In one aspect, the invention provides a method of inhibiting the growth of lymphoma cells, suitably non-Hodgkin's lymphoma cells, that are not responsive to GaN by contacting the cells with an effective amount of a gallium (III) complex or derivatives thereof to inhibit cell growth. A suitable gallium (III) complex is gallium maltolate (GaM).

In another aspect, the invention provides a method of treating a mammal having a tumor that is not responsive to GaN by administering to the mammal a therapeutically effective amount of a gallium (III) complex or a derivative thereof to treat the tumor. GaN resistant tumors include lymphomas and suitably non-Hodgkin's lymphomas.

In another aspect, the gallium (III) complex, suitably GaM may be administered orally, parenterally, or intravenously either alone or in combination with other chemotherapeutic agents to treat GaN resistant tumors.

Unless otherwise defined, all technical and scientific terms used herein have the same meaning as commonly understood by one of ordinary skill in the art to which this invention belongs. Although suitable methods and materials for the practice or testing of the present invention are described below, other methods and materials similar or equivalent to those described herein, which are well known in the art, can also be used.

Other objects, advantages and features of the present invention will become apparent from the following specification taken in conjunction with the accompanying drawings.

### BRIEF DESCRIPTION OF THE SEVERAL VIEWS OF THE DRAWINGS

FIG. 1 is a structure of gallium maltolate (GaM), a neutral 3:1 (hydroxypyrone:gallium) complex wherein the hydroxypyrone is 3-hydroxy-2-methyl-4-pyrone.

FIG. 2 shows that inhibition of CCRF-CEM cell growth by GaM is due to gallium and not maltol.

FIG. 3 is a comparison of the cytotoxic effects of GaN and GaM in mantle cell lymphoma HBL-2 cells.

FIG. 4 shows that gallium maltolate activates caspase-3 at lower gallium concentrations than gallium nitrate in HBL-2 cells.

FIGS. 5A-D show that CCRF-CEM cells resistant to gallium nitrate are sensitive to GaM: (A) depicts the effect of gallium nitrate on the growth of gallium nitrate-resistant (GnR) and -sensitive (GnS) cells, (B) depicts the effect of GaM on the growth of gallium nitrate-resistant (GnR) and -sensitive (GnS) cells, (C) depicts a comparison of the effects of GaM and GaN on GnR cells, (D) is a graph showing GaM, but not GaN, activates caspase-3 in GnR cells resulting in apoptosis.

FIGS. 6A-C show that GaM inhibits the growth of cells independent of p53 status: (A) p53 wt TK-6 cells, (B) p53 null HN-32 cells, (C) p53 mutant WTK-1 cells.

FIG. 7 shows GaM inhibits cellular iron uptake. ⁵⁸Fe-Tf uptake by HBL-2 cells (0.2×10⁶) was measured.

FIG. 8 shows the effect of GaM on HBL-2 cell surface transferrin (Tf) receptor density. Maximum Tf binding was determined by Scatchard analysis of ¹²⁵I-Tf binding to intact cells at 4° C.

FIG. 9 is a tabular comparison of GaM and GaN IC₅₀s for a panel of lymphoma cell lines, including CCRF-CEM GaN-resistant cells, mantle cell lymphoma (MCL) cells, and p53 variant lymphoma cells.

FIG. 10A-B show that cellular uptake of gallium is greater with GaM than with GaN: (A) sensitive and (B) resistant (non-responsive) cells were incubated with increasing concentrations of gallium nitrate or gallium maltolate containing Ga-67 as a tracer.

### DETAILED DESCRIPTION OF THE INVENTION

The present invention broadly relates to a novel method for inhibiting the growth of a diverse array of gallium nitrate-resistant tumor cells in mammals by contacting such cells with an effective amount of a gallium-containing compound. The invention is based upon the discovery that certain tumor cells are not responsive to GaN, but are responsive to other gallium (III) complexes. Accordingly, the methods of the invention broadly relate to using a gallium (III) complex or derivatives thereof described herein to effectively inhibit growth of tumor cells not responsive to GaN.

In one embodiment, the invention provides a method of inhibiting the growth of lymphoma cells resistant to GaN by contacting the cells with an effective amount of a gallium (III) complex or derivatives thereof to inhibit cell growth. A suitable gallium (III) complex is gallium maltolate (GaM).

In one embodiment, the invention provides a method of inhibiting the growth of non-Hodgkin's lymphoma cells resistant to GaN by contacting the cells with an effective amount of GaM to inhibit cell growth.

In another embodiment, the invention provides a method of treating a mammal having a tumor not responsive to GaN by administering to the mammal a therapeutically effective amount of a gallium (III) complex, resulting in the effective therapeutic treatment of the tumor.

In a preferred embodiment, the tumor is a lymphoma, suitably a non-Hodgkin's lymphoma that is not responding to GaN treatment. A suitable gallium (III) complex may be administered orally, parenterally, or intravenously either alone or in combination with other chemotherapeutic agents. Other chemotherapeutic agents may include other inhibitors of ribonucleotide reductase or DNA synthesis, which could bring about a synergistic antitumor effect against lymphoma patients who are not responding to treatment with GaN.

In yet another embodiment, the invention provides a method of inhibiting GaN resistant lymphoma cells by contacting the cells with an effective amount of GaM sufficient to inhibit cell growth. In a preferred embodiment, the cells are GaN resistant non-Hodgkin's lymphoma cells.

For convenience, certain terms employed in the specification, examples, and appended claims are provided here.

As used herein, the term “gallium-containing compound” broadly encompasses compounds with a semi-metallic element (gallium) that are potent inhibitors of ribonucleotide reductase, an enzyme that promotes tumor growth. As used herein, suitable gallium-containing compounds include a coordination complex of gallium (III), an organic salt of gallium (III), or peptide or protein-bound gallium (III).

As used herein, exemplary gallium (III) coordination complexes may include N-heterocycle complexes (e.g., tris (8-quinolinolato) gallium (III)), complexes with hydroxypyrones including neutral 3:1 gallium (III) complexes such as gallium maltolate, hydroxypyridinones or substituted hydroxypyridinones gallium (III) complexes, gallium porphyrins (e.g., gallium (III) protoporphyrin IX), pyridoxal isonicotinoyl hydrazone gallium (III), and gallium salt complexes of polyether acids. These coordination complexes include and are not limited to three bidentate ligands or one tridentate ligands.

Furthermore, gallium (III) coordination complexes may encompass neutral 3:1 hydroxypyrone:gallium complexes, wherein the hydroxypyrone may be substituted, unsubstituted, branched, linear, saturated or unsaturated. There may also be alkyls from 0 to 6 carbon atoms at positions C2 and C6 on the hydroxypyrone. Also, all of the pharmaceutical compositions comprising gallium (III) complexes of 3-hydroxy-4-pyrones disclosed in U.S. Patent Nos. 6,087,354 and 6,004,951 are incorporated by reference herein in their entirety.

A preferred gallium-containing compound is a neutral 3:13-hydroxy-4-pyrone:gallium complex, wherein the hydroxypyrone is 3-hydroxy-2-methyl-4-pyrone.

As used herein, exemplary gallium (III) organic salts could include gallium acetate, gallium citrate, gallium formate, gallium hydroxamate, gallium oxalate, gallium glutamate, gallium palmitate, and gallium tartrate, as well as their hydrated and solvated forms.

As used herein, exemplary protein-bound compositions could include gallium-lactoferrin or gallium-transferrin or gallium-transferrin-doxorubicin (or other similar toxin) conjugates.

In practicing the invention, gallium-containing compounds, such as GaM may be used to inhibit growth of cancer cells, suitably lymphoma cells, and more suitably non-Hodgkin's lymphoma cells, that are not responsive to GaN. Such growth inhibition is possible because GaM as described herein inhibits cell growth and induces apoptosis at significantly lower concentrations than GaN. Notably, GaM induces apoptosis in cell lines resistant to GaN. GaM activates Caspase-3, but the steps leading to this have yet to be determined. (See, Chitambar C R, et al. (2007) Mol Cancer Ther, 6(2):633-643, each incorporated by reference here in its entirety). GaM is active against cell lines that are resistant to GaN and acts independently of p53 status. GaM interferes with cellular iron uptake and alters transferrin receptor expression. An oral formulation of a GaM compound with increased bioavailability for administration to mammals with lymphoma is preferred.

Although, the mechanism for GaM is yet unknown, in general gallium's mechanisms of action include its binding to transferrin, targeting to transferrin receptors on lymphoma cells, and inhibiting ribonucleotide reductase. Recent investigations show that gallium activates caspases and induces apoptosis through the mitochondrial pathway, whereas complementary DNA microarray studies suggest that changes in intracellular trafficking pathways may be important in gallium resistance.

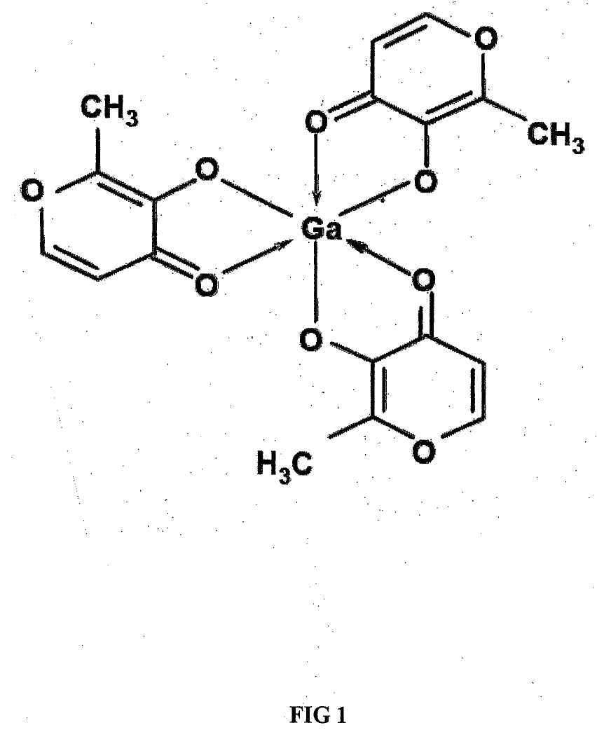

In a preferred embodiment GaM, as disclosed in FIG. 1 is orally administered to a mammal, suitably a human afflicted with a lymphoma that is not responsive to GaN. In this embodiment, the orally active formulation of gallium, GaM, provides advantages of enhanced bioavailability, a potentially improved therapeutic profile and ease of administration compared to GaN. It is contemplated that an oral formulation of GaM will provide a good safety profile, with attainment of targeted therapeutic serum drug levels, and pharmacokinetics that support twice a day or once a day dosing.

The term “lymphoma” as used herein refers to cancer of the lymph nodes. Lymphoma encompasses low grade non-Hodgkin's Lymphoma, intermediate grade non-Hodgkin's Lymphoma, follicular lymphoma, large cell lymphoma, B-cell lymphoma, T-cell lymphoma, Mantle cell lymphoma, Burkitt's lymphoma, NK cell lymphoma, acute lymphoblastic lymphoma, and lymphoma cells that are resistant to gallium nitrate. The physiological condition of a mammal diagnosed with this disease is typically characterized by unregulated increase in cell number (generally referred to herein as cell growth), which can be due to abnormal increase in cell proliferation, abnormal decrease of cell death (apoptosis), or an imbalance of amounts of cell proliferation and cell death.

The term “non-Hodgkin's lymphoma” or “NHL”, as used herein, refers to a cancer of the lymphatic system other than Hodgkin's lymphomas. Hodgkin's lymphomas can generally be distinguished from non-Hodgkin's lymphomas by the presence of Reed-Stemberg cells in Hodgkin's lymphomas and the absence of said cells in non-Hodgkin's lymphomas. Examples of non-Hodgkin's lymphomas encompassed by the term as used herein include any that would be identified as such by one skilled in the art (e.g., an oncologist or pathologist) in accordance with classification schemes known in the art, such as the Revised European-American Lymphoma (REAL) scheme as described in Color Atlas of Clinical Hematology, Third Edition; A. Victor Hoffbrand and John E. Pettit (eds.) (Harcourt Publishers Limited 2000). More specific examples include, but are not limited to, relapsed or refractory NHL, front line low grade NHL, Stage III/IV NHL, chemotherapy resistant NHL, precursor B lymphoblastic leukemia and/or lymphoma, small lymphocytic lymphoma, B cell chronic lymphacytic leukemia and/or prolymphocytic leukemia and/or small lymphocytic lymphoma, B-cell prolymphocytic lymphoma, immunocytoma and/or lymphoplasmacytic lymphoma, marginal zone B cell lymphoma, splenic marginal zone lymphoma, extranodal marginal zone—MALT lymphoma, nodal marginal zone lymphoma, hairy cell leukemia, plasmacytoma and/or plasma cell myeloma, low grade/follicular lymphoma, intermediate grade/follicular NHL, mantle cell lymphoma, follicle center lymphoma (follicular), intermediate grade diffuse NHL, diffuse large B-cell lymphoma, aggressive NHL (including aggressive front-line NHL and aggressive relapsed NHL), NHL relapsing after or refractory to autologous stem cell transplantation, primary mediastinal large B-cell lymphoma, primary effusion lymphoma, high grade immunoblastic NHL, high grade lymphoblastic NHL, high grade small non-cleaved cell NHL, bulky disease NHL, Burkitt's lymphoma, precursor (peripheral) T-cell lymphoblastic leukemia and/or lymphoma, adult T-cell lymphoma and/or leukemia, T cell chronic lymphocytic leukemia and/or prolymphacytic leukemia, large granular lymphocytic leukemia, mycosis fungoides and/or Sezary syndrome, extranodal natural killer/T-cell (nasal type) lymphoma, enteropathy type T-cell lymphoma, hepatosplenic T-cell lymphoma, subcutaneous panniculitis like T-cell lymphoma, skin (cutaneous) lymphomas, anaplastic large cell lymphoma, angiocentric lymphoma, intestinal T cell lymphoma, peripheral T-cell (not otherwise specified) lymphoma and angioimmunoblastic T-cell lymphoma.

An “effective amount” or a “therapeutically effective amount” of GaM as disclosed herein is an amount sufficient to inhibit lymphoma cell growth. An “effective amount” may be determined empirically and in a routine manner, in relation to the stated purpose. More specifically, the term “therapeutically effective amount” refers to an amount of GaM effective to “treat” a disease or disorder in a subject or mammal. In the case of non-Hodgkin's Lymphoma, the therapeutically effective amount of the compound may reduce the number of affected cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. See the definition herein of “treating”. To the extent the GaM compound may prevent growth and/or kill existing cancer cells, it may be cytostatic and/or cytotoxic.

A growth inhibitory amount of a GaM compound is an amount capable of inhibiting the growth of a cell, especially tumor, e.g., cancer cell, preferably lymphoma, either in vitro or in vivo. Inhibition of neoplastic cell growth may be determined empirically and in a routine manner.

The term “inhibit” or “inhibiting” means decreasing tumor cell growth rate from the rate which would occur without treatment, and/or causing tumor mass to decrease. Inhibiting also includes causing a complete regression of the tumor. Thus, the present analogs can either be cytostatic or cytotoxic to tumor cells.

As used herein the term “mammal” is for purposes of the treatment of, alleviating the symptoms of a cancer refers to any animal classified as a mammal, including humans, domestic and farm animals, and zoo, sports, or pet animals, such as dogs, cats, cattle, horses, sheep, pigs, goats, rabbits, etc. Preferably, the mammal is human.

In one embodiment, a suitable human that would benefit from the method of the invention is a patient with non-Hodgkin's lymphoma, who has had their disease relapse after treatment with conventional antineoplastic agents, such as chemotherapy. In a preferred embodiment, the patient is one whose lymphoma has not responded to treatment with GaN, but would respond to GaM. Accordingly, the invention described here shows that lymphoma cell lines that do not respond to gallium nitrate (i.e., are resistant to gallium nitrate) do surprisingly respond to GaM.

As used herein the terms “treating” or “treatment” refer to both therapeutic treatment and prophylactic or preventative measures, wherein the object is to prevent or slow down (lessen) the targeted pathologic condition or disorder. Those in need of treatment include those already with the disorder as well as those prone to have the disorder or those in whom the disorder is to be prevented.

A subject or mammal is successfully “treated” for a non-Hodgkin's lymphoma if, after receiving a therapeutic amount of GaM according to the methods of the invention, the patient shows observable and/or measurable reduction in or absence of one or more of the following: reduction in the number of cancer cells or absence of the cancer cells; reduction in the tumor size; inhibition (i.e., slow to some extent and preferably stop) of cancer cell infiltration into peripheral organs including the spread of cancer into soft tissue and bone; inhibition (i.e., slow to some extent and preferably stop) of tumor metastasis; inhibition, to some extent, of tumor growth; and/or relief to some extent, one or more of the symptoms associated with the specific cancer; reduced morbidity and mortality, and improvement in quality of life issues. To the extent the GaM may prevent growth and/or kill existing cancer cells, it may be cytostatic and/or cytotoxic. Reduction of these signs or symptoms may also be felt by the patient.

The above parameters for assessing successful treatment and improvement in the disease are readily measurable by routine procedures familiar to a physician. For cancer therapy, efficacy can be measured, for example, by assessing the time to disease progression (TTP) and/or determining the response rate (RR). Metastasis can be determined by staging tests and by bone scan and tests for calcium level and other enzymes to determine spread to the bone. CT scans can also be done to look for spread to the pelvis and lymph nodes in the area. Chest X-rays and measurement of liver enzyme levels by known methods are used to look for metastasis to the lungs and liver, respectively. Other routine methods for monitoring the disease include transrectal ultrasonography (TRUS) and transrectal needle biopsy (TRNB).

In another embodiment, it is contemplated that GaM is administered in a pharmaceutical composition containing a pharmaceutically acceptable carrier. Such a carrier includes pharmaceutically acceptable carriers, excipients, or stabilizers which are nontoxic to the cell or mammal being exposed thereto at the dosages and concentrations employed. A carrier may be liquid or solid and suitable for oral administration. Examples of physiologically acceptable carriers include buffers; antioxidants; low molecular weight polypeptide; proteins; hydrophilic polymers; amino acids; carbohydrates; chelating agents; sugar alcohols; salt-forming counterions; and/or nonionic surfactants.

In a preferred embodiment, the pharmaceutical composition may be in the form of a tablet or capsule. The GaM pharmaceutical composition may include an additional active agent. It is also envisioned that the GaM pharmaceutical composition maybe co-administered with other compounds for the treatment cancer, preferably lymphomas described herein.

As used herein, the term “administration” means alone or “in combination with” one or more further therapeutic agents including simultaneous (concurrent) and consecutive administration in any order. In addition to oral administration, it is contemplated that the GaM compound may be administered to humans for therapy by other suitable routes of administration, including nasally, as by, for example, a spray, rectally, intravaginally, parenterally (intravenous, subcutaneous, or intramuscular injection), intracistemally and topically, as by powders, ointments or drops, including buccally and sublingually.

Regardless of the route of administration selected, the GaM compounds of the present invention may be used in a suitable hydrated form, and/or pharmaceutical composition formulated into pharmaceutically-acceptable dosage forms, such as described below or by other conventional methods known to those of skill in the art.

The selected and actual dosage levels of the GaM pharmaceutical compositions of this invention can be varied to obtain an amount of the active ingredient effective to achieve the desired therapeutic response for a particular individual, composition, and mode of administration, without being toxic to the patient.

Accordingly, it is envisioned that the dosage levels will vary depending upon a variety of factors. Such factors include the activity of the GaM compound employed, or the ester, salt or amide thereof, the route of administration, the time of administration, the rate of excretion of the particular compound being employed, the duration of the treatment, other drugs, compounds and/or materials used in combination with the particular apoptosis-inducing agent employed, the age, sex, weight, condition, general health and prior medical history of the patient being treated, and like factors well known in the medical arts. A physician or veterinarian having ordinary skill in the art can readily determine and prescribe the effective amount of the pharmaceutical composition required.

In general, a suitable daily dose of a gallium (III) complex, suitably GaM compound of the invention will be that amount of the compound which is the lowest dose effective to produce a therapeutic effect. Such an effective dose will depend upon the factors described above.

It is envisioned that typically gallium-containing compound will be administered as a single or multiple dose daily to provide a total daily amount of elemental gallium of about 2 mg to about 800 mg. Suitable daily dose ranges include but are not limited to about 2-15 mg, 8-40 mg, 15-80 mg, 40-160 mg, 150-325 mg, 300-500 mg, 500-700 mg, and 600-800 mg. As discussed herein the actual dosage may vary depending upon the gallium compound being administered, its formulation and bioavailability, and the method of administration.

For example, with regard to GaM, it is contemplated that doses range from about 10 mg/day to about 5 g/day when examined in human subjects with tumors resistant to GaN. In contrast and as a comparison, it is noted that GaN is typically only administered intravenously and the recommended dose ranges from 200 to 400 mg/m²/day for a 7 day period to treat lymphoma. (See Chitambar, C. (2004), incorporated by reference here in its entirety.)

It is to be understood that this invention is not limited to the particular methodology, protocols, cell lines, animal species, and reagents described, as such may vary. It is also to be understood that the terminology used herein is for the purpose of describing particular embodiments only, and is not intended to limit the scope of the present invention which will be limited only by the appended claims.

### EXAMPLES

### Materials and Methods

CCRF-CEM cells were obtained from ATCC. The gallium nitrate resistant CCRF-CEM cell line was developed by the inventor, as previously reported (Chitambar C R, Wereley J P, (1997) “Resistance to the antitumor agent gallium nitrate in human leukemic cells is associated with decreased gallium/iron uptake, increased activity of iron regulatory protein-1, and decreased ferritin production.” J Biol Chem; 272:12151-7; Chitambar C R, et al. (1996) Clinical Cancer Research, 2:1009-1015; Chitambar C R, Wereley J P, (1998) Blood, 91(12): 4686-4693; and Chitambar C R, et al. (2007) Mol Cancer Ther, 6(2):633-643, each incorporated by reference here in its entirety).

Mantle cell lymphoma (HBL-2 (Human B-cell lymphoma-derived cell line), Granta, JVM-2, NCEB-1, Z138C) and DoHH2 cell lines were obtained from the British Columbia Cancer Agency. p53 cell lines were obtained from the NCI. Gallium maltolate and gallium nitrate were obtained from Titan Pharmaceuticals Inc. (San Francisco, Calif.) and Genta Incorporated (Berkley Heights, N.J.), respectively. Cell proliferation was measured by MTT assay (Mosmann T. “Rapid colorimetric assay for cellular growth and survival: Application to proliferation and cytotoxicity assays”. J Immunol Methods 1983; 65:55-63, incorporated by reference here in its entirety). Caspase-3 activity was measured by ApoOne assay (Promega, Madison, Wis.). Assays for cellular ⁵⁹Fe-transferrin uptake and ¹²⁵I-transferrin binding were used as previously described in Chitambar, 1997.

### Example 1

### Inhibition of CCRF-CEM Cell Growth by GaM is Due to Gallium and not Maltol

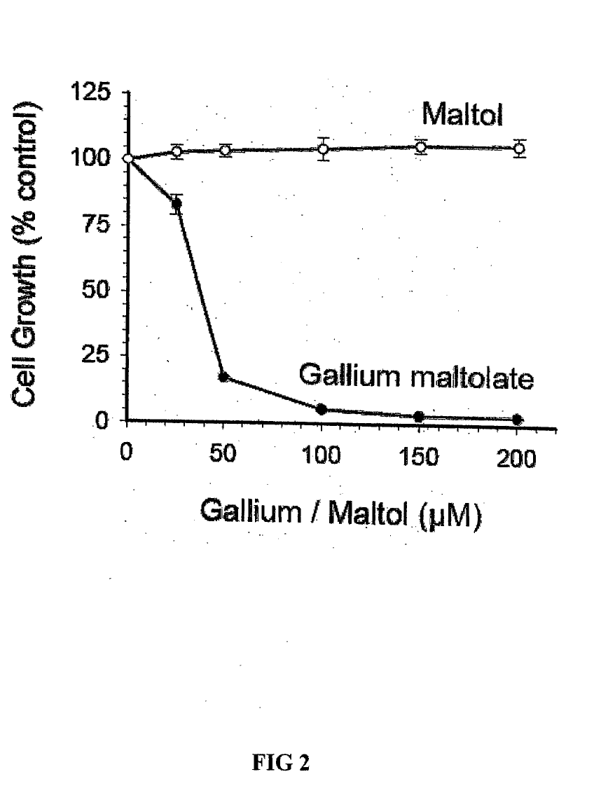

CCRF-CEM cells were plated at a density of 2×10⁵ cells/ml in microwell plates (100 μL cell suspension per well) in the presence of increasing concentrations of gallium maltolate or maltol (subsequently referred to as the additives). After 72 h of incubation, 10 μL MTT (3-(4,5-dimethlythiazol-2-yl)-2,5-diphenyltetrazolium bromide) was added to each well and the incubation continued for 4 h at 37° C. Cells were then solubilized by the addition of 100 μL of 0.4 N HCl in isopropanol to each well and the absorbance of each well was read at 570 nm using a microplate reader. The effect of the additives on cell proliferation was determined by comparing the absorbance from wells containing the additives with the absorbance from wells in which the additives were omitted (decreased absorbance signifies decreased cell proliferation). Cell growth was expressed as a percentage of control (cell growth in the absence of additives). Cell proliferation was assessed after 72 h of incubation with gallium maltolate or maltol. Maltol alone did not inhibit cell growth indicating that the antineoplastic effects of GaM were due to gallium per se and not maltol. Thus, the results show that cell growth was inhibited by gallium maltolate but not by maltol as depicted in FIG. 2.

### Example 2

### Comparative Effects of GaN and GaM in Mantle Cell Lymphoma HBL-2 Cells

HBL-2 cells were plated at a density of 2×10⁵ cells/ml in microwell plates (100 μL cell suspension per well) in the presence of increasing concentrations of GaM or GaN (referred to as the additives). After 72 h of incubation, 10 μL MTT (3-(4,5-dimethlythiazol-2-yl)-2,5-diphenyltetrazolium bromide) was added to each well and the incubation continued for 4 h at 37° C. Cells were then solubilized by the addition of 100 μL 0.4 N HCl in isopropanol to each well and the absorbance of each well was read at 570 nm using a spectrophotometer. Cell proliferation was measured after a 72 h incubation period.

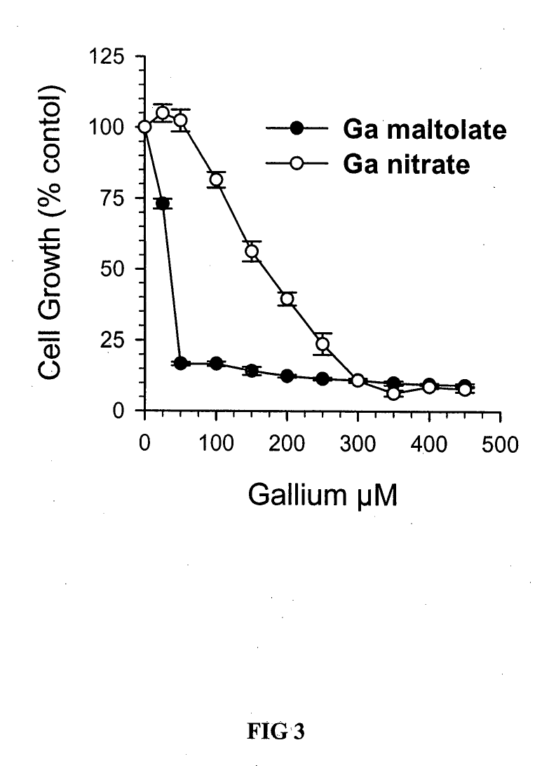

The effect of the additives on cell proliferation was determined by comparing the absorbance from wells with the additives with the absorbance from wells in which the additives were omitted (decreased absorbance signifies decreased cell proliferation). Cell growth was expressed as a percentage of control (cell growth in the absence of additives). The results depicted in FIG. 3 show that when gallium was presented to cells as GaM, much lower concentrations of gallium were required to inhibit cell proliferation than when gallium was presented as gallium nitrate.

### Example 3

### GaM Activates Caspase-3 at Lower Gallium Concentrations than GaN in HBL-2 Cells

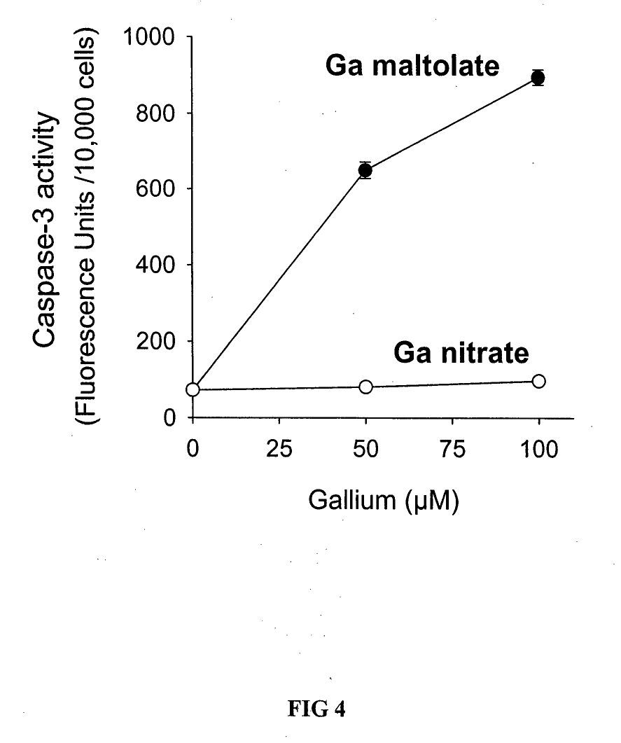

HBL-2 cells were incubated with increasing concentrations of GaM or GaN for 48 h. After incubation with these compounds, the cells were analyzed for apoptosis using the commercially available apo-One™ assay that measures caspase-3 activation. Data points are the means and S.E. of an experiment performed in triplicate. The experimental results depicted in FIG. 4 show that caspase-3 is activated with GaM but not with similar concentrations of GaN.

### Example 4

### CCRF-CEM Cells Resistant to GaN are Sensitive to GaM

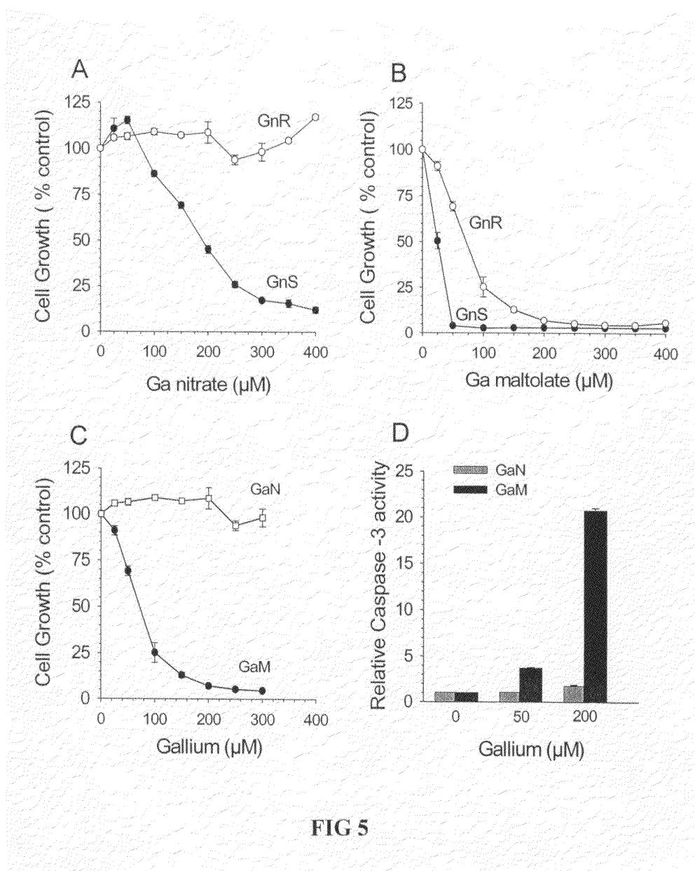

The CCRF-CEM lymphoma cell line resistant (GnR) to GaN developed in the inventor's laboratory was used as a model to test sensitivity of GaN resistant cells to GaM. To determine the effect of gallium nitrate on the growth of gallium nitrate-resistant (GnR) and -sensitive (GnS) cells, cells were plated in microwell plates and incubated with gallium nitrate. Then cellular proliferation was measured by MTT assay after 72 h incubation with gallium nitrate. Cell growth was determined by MTT assay as described in Example 1 above. Values shown are means +/−S.E. (n=4). The results are shown in FIG. 5A.

To determine the effect of GaM on the growth of gallium nitrate-resistant (GnR) and -sensitive (GnS) cells, cells were plated in microwell plates and incubated with GaM. Cellular proliferation was measured by MTT assay after 72 h incubation with GaM. Values shown are means +/−S.E. (n=4). The results are shown in FIG. 5B.

To compare the effects of GaM and gallium nitrate on GnR cells, cell growth was measured by MTT assay after 72 h incubation. The results are shown in FIG. 5C.

Next, GnR cells were incubated for 24 h with GaM or GaN at the concentrations shown in FIG. 5D. After the incubation period, caspase-3 activity was measured by apo-One™ assay. FIG. 5D shows that GaM inhibits proliferation and activates caspase-3 in GnR cells resistant to GaN.

### Example 5

### GaM Inhibits the Growth of Cells Independent of p53 Status

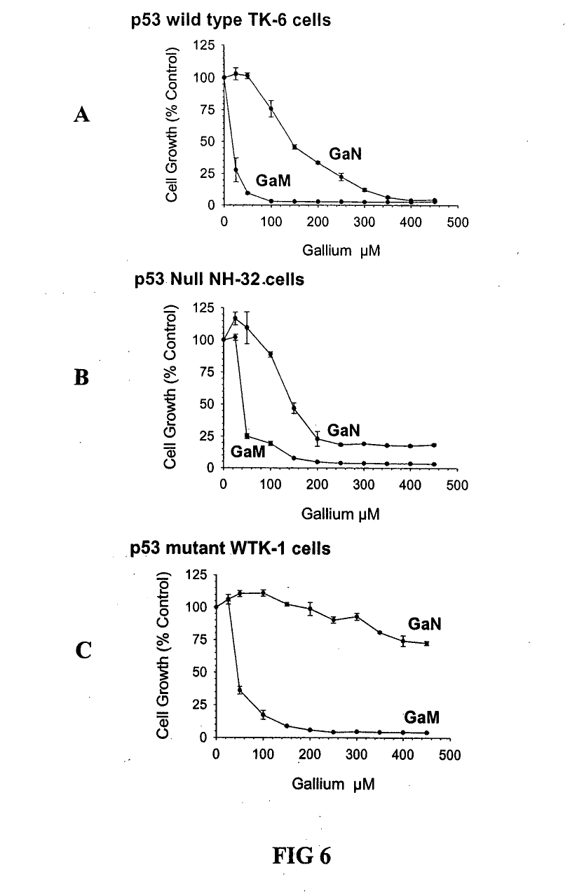

The effect of GaN and GaM on human B lymphoblast cells lines with different p53 expression was examined. Cell growth was measured by MTT assay as described in the experimental details for Example 1. Cell growth was determined after 72-h incubation of cells with increasing concentrations of GaM (open circles) or GaN (closed circles). Values shown represent the means±S.E. (n=3). FIG. 6 shows that the growth of all three cell lines, regardless of p53 status, was inhibited to a similar extent by GaM. In contrast, the growth of p53 mutant WTK-1 cells (containing a methionine to isoleucine substitution at codon 237) was not inhibited by GaN.

### Example 6

### GaM Inhibits Cellular Iron Uptake

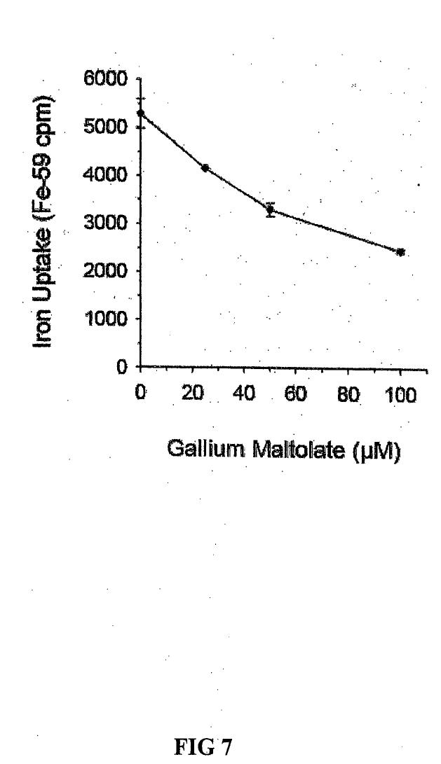

HBL-2 cells were incubated in complete medium in the presence of increasing concentrations of GaM. Radioiron (⁵⁹Fe-transferrin) was added to cells at the start of the incubation. After 24 h, cells were harvested, washed with phosphate buffered saline to remove unincorporated ⁵⁹Fe and centrifuged to pellet the cells. ⁵⁹Fe radioactivity in the cell pellet was counted in a gamma counter. The results showing that the amount of radioiron taken up by cells decreases with increasing concentrations of GaM are depicted in FIG. 7.

### Example 7

### Effect of GaM on HBL-2 Cell Surface Transferrin (Tf) Receptor Density

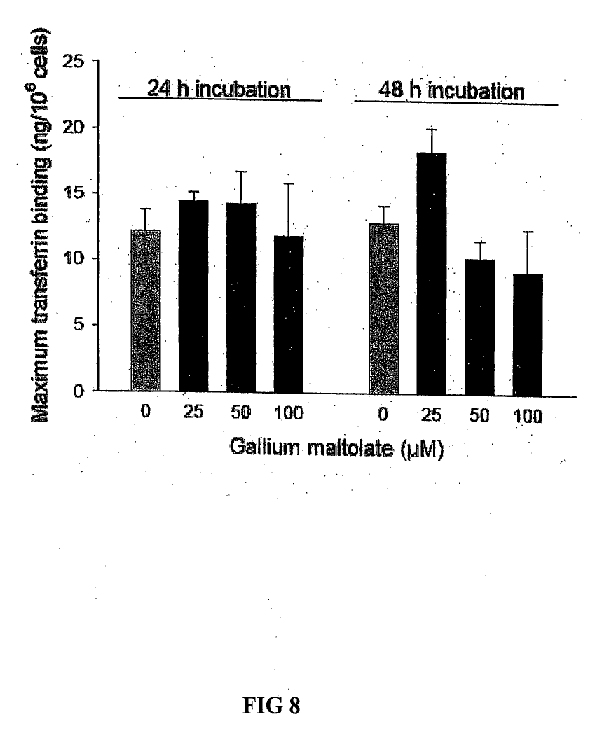

To determine the mechanism by which GaM acts on cells, cellular transferrin receptor expression was evaluated in HBL-2 cells. The effect of GaM on cellular transferrin receptor expression was determined using a ¹²⁵I-transferrin (Tf) binding assay that measures the binding of the ligand Tf to its receptor (TfR). HBL-2 cells were incubated with increasing concentrations of GaM for 24 and 48 h and then analyzed for Tf binding using an established method. The amount of radioactive Tf bound to cellular TfR was calculated using Scatchard analysis. FIG. 8 shows that at 24 h, 25 and 50 μM GaM increase the number of TfRs present on the cell surface while 48 h, 25 μM GaM increases TfR number. Since TfR levels are physiologically regulated by cellular iron status, the change in TfR by GaM suggests that it interferes with cellular iron homeostasis.

### Example 8

### Comparison of GaM and GaN IC₅₀s for a Panel of Lymphoma Cell Lines

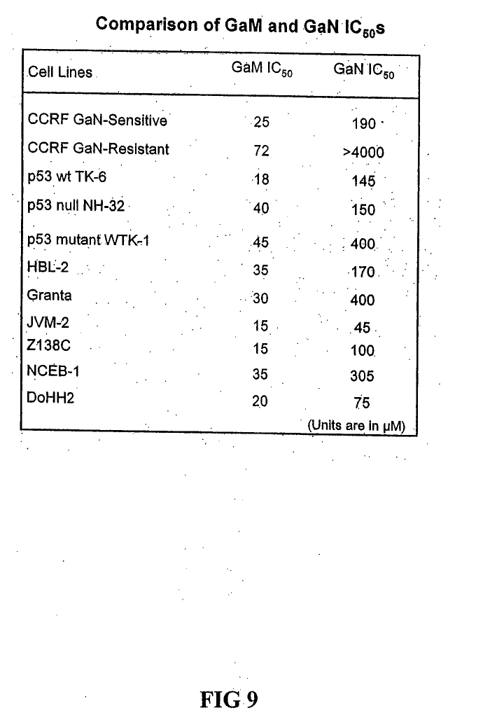

The effects of GaM and GaN on the growth of a panel of lymphoma cell lines that include unique CCRF-CEM GaN-resistant cells, mantle cell lymphoma (MCL) cells, and p53 variant cells shown in FIG. 9 were determined by MTT assay, as described in the Experimental Details for Example 1. Cell growth was determined after 72 h of incubation of cells with GaM or GaN.

It was determined that both GaN and GaM inhibited T lymphoma CCRF-CEM cell growth with IC50s of 103 μM (GaN) and 29 μM (GaM), respectively. Whereas GaN did not inhibit the growth of GaN-resistant cells, even at 4000 μM GaN, their growth was inhibited by GaM (IC50 70 μM). The growth of MCL cell lines HBL2, JVM2, Z138C, and Granta was inhibited by GaN at IC50s of 45-400 μM.

In contrast, the IC50 of GaM for these cell lines was 15-30 μM. GaN IC50s for p53 wt TK-6 and p53 null HN-32 cells were 145 and 150 μM respectively, while the GaM IC50s were 18 and 45 μM. The growth of p53 mutant WTK-1 cells was not inhibited by GaN (IC50 >400 μM) but was inhibited by GaM (IC50 45 μM). Cells exposed to either GaN or GaM displayed an increase in Annex in V staining and a dose-dependent increase in caspase-3 activity; however, caspase-3 activation occurred with lower concentrations of GaM than with GaN (see FIG. 4).

Applicants surprisingly discovered that GaM has significant activity against a panel of B- and T-cell lymphoma cell lines. The IC50 of GaM is significantly lower than that of GaN in all the cell lines examined. Accordingly, FIG. 9 shows that in every cell line examined, the growth-inhibitory effects of GaM were greater than that of GaN.

This result was unexpected and unpredictable.

Furthermore, both GaN and GaM display cytotoxicity against NHL cells; however, GaM inhibits cell growth and induces apoptosis at significantly lower concentrations than GaN. Notably, GaM induces apoptosis in cell lines resistant to GaN. This suggests that although the two gallium compounds may have certain intracellular targets in common, GaM targets additional processes, thus circumventing drug resistance to GaN. The inventor is conducting ongoing mechanism studies of apoptotic activity of GaM. This information will be important in identifying lymphoma subtypes that may be responsive to treatment with this GaM or its derivatives.

In accordance with the present invention further studies in animal tumor models are warranted to evaluate the antineoplastic activity of GaM in vivo. It is contemplated, however, that the significant activity and the antineoplastic effects of GaM on the experimental panel of B- and T-cell lymphoma cell lines is highly predictive of lymphoma cells in vivo. The cell lines described herein can be readily correlated with in vivo results for treatment of lymphoma and related diseases.

### Example 9

### Cellular Gallium Uptake is More Efficient with Gam

To evaluate cellular uptake of GaM and GaN in cells, the gallium-sensitive and -resistant CCRF-CEM cells developed by the inventor were plated (5×10⁵ cells/mL) in 1-ml multiwell plates and incubated with increasing concentrations of either GaM or GaN containing ⁶⁷Ga as a tracer (1 μCi of ⁶⁷Ga/1 mM GaM or GaN). After 24 h of incubation, the number of cells in each well was counted with a hemocytometer and the cells were harvested and washed by centrifugation with phosphate-buffered saline. ⁶⁷Ga radioactivity in the cell pellet was counted in a gamma counter and the amount of ⁶⁷Ga incorporated per 10⁶ cells determined.

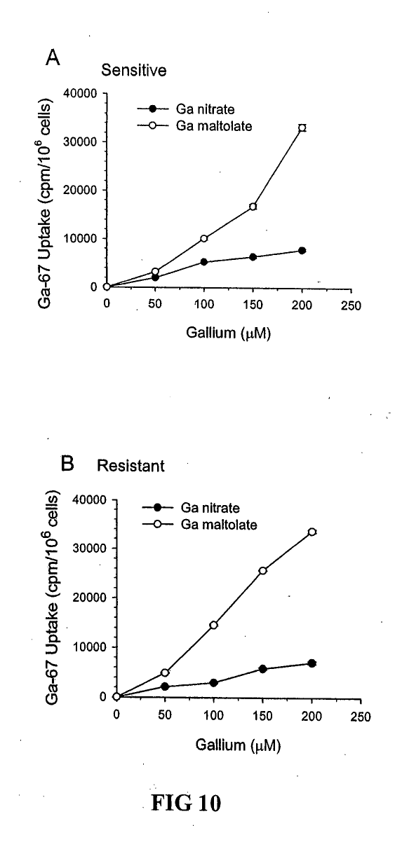

To examine whether differences in the cytotoxicity of GaM and GaN could be related to differences in their ability to transport gallium into cells, the cellular uptake of gallium was examined using ⁶⁷Ga as a tracer for either gallium compound. As shown in FIG. 10, over a 24 h incubation, both gallium-sensitive and -resistant cells incorporated greater amounts of ⁶⁷Ga as GaM than as GaN. These results suggest that the uptake of gallium is more efficient when gallium is presented to cells as GaM. The results also help to advance understanding of the mechanism action for GaN resistance by some lymphoma cells. The implication is that GaM is able to get more gallium into cells and bypass yet unknown steps related to gallium nitrate resistance. Overall, the results depicted in FIG. 10 show that the uptake of gallium is greater when gallium is presented to cells as GaM rather than as GaN.

The disclosure of every patent, patent application, and publication cited herein is hereby incorporated herein by reference in its entirety.

It is understood that certain adaptations of the invention described in this disclosure are a matter of routine optimization for those skilled in the art, and can be implemented without departing from the spirit of the invention, or the scope of the appended claims.

We claim:

1. A method of inhibiting gallium nitrate-resistant tumor cell growth in a mammal comprising contacting the cell with an effective amount of gallium-containing compound to inhibit the cell growth.

2. The method of claim 1, wherein the gallium-containing compound is a coordination complex of gallium (III), an organic salt of gallium (III), or peptide or protein-bound gallium (III).

3. The method of claim 2, wherein the coordination complex of gallium (III) is a 3:1 hydroxypyrone:gallium complex.

4. The method of claim 2, wherein the complex is gallium maltolate (GaM).

5. The method of claim 1, wherein the tumor cell is a lymphoma cell that is non-responsive to gallium nitrate.

6. The method of claim 5, wherein the lymphoma cell is a non-Hodgkin's lymphoma cell that is non-responsive to gallium nitrate.

7. The method of claim 1, wherein the mammal is a human.

8. A method of inhibiting gallium nitrate-resistant lymphoma cell growth in a mammal comprising contacting the cell with an effective amount of gallium-containing compound to inhibit the cell growth.

9. The method of claim 8, wherein the lymphoma cell is a non-Hodgkin's lymphoma cell that is non-responsive to gallium nitrate.

10. The method of claim 8, wherein the gallium-containing compound is a coordination complex of gallium (III), an organic salt of gallium (III), or peptide or protein-bound gallium (III).

11. The method of claim 10, wherein the coordination complex of gallium (III) is a 3:1 hydroxypyrone:gallium complex.

12. The method of claim 11, wherein the complex is gallium maltolate (GaM).

13. A method of treating a gallium nitrate-resistant tumor in a mammal, the method comprising administering to the mammal an effective amount of a gallium-containing compound to treat the tumor.

14. The method of claim 13, wherein the gallium-containing compound is a coordination complex of gallium (III), an organic salt of gallium (III), or peptide or protein-bound gallium (III).

15. The method of claim 14, wherein the coordination complex of gallium (III) is gallium maltolate (GaM).

16. The method of claim 13, wherein the tumor is a lymphoma that is non-responsive to gallium nitrate.

17. The method of claim 16, wherein the lymphoma is a non-Hodgkin's lymphoma cell that is non-responsive to gallium nitrate.

18. The method of claim 13, wherein the gallium-containing compound is administered orally, parenterally, or intravenously.

19. The method of claim 18, wherein the gallium-containing compound is administered as a single or multiple dose daily to provide a total daily amount of elemental gallium of about 2 mg to about 800 mg

20. The method of claim 18, wherein GaM is administered orally in therapeutically effective amounts ranging from about 10 mg/day to about 5 grams/day in humans.

21. The method of claim 13, wherein the gallium containing compound is administered in combination with other chemotherapeutic agents.

22. A method of treating gallium-nitrate resistant lymphoma in a human, the method comprising administering to the mammal an effective amount of a GaM to treat the lymphoma.
